(19) United States
(12) Patent Application Publication
(10) Pub. No.: US 2003/0187058 A1
(43) Pub. Date: Oct. 2, 2003

(54) Combination of lipoic acids and conjugated acids for treating diabetic disorders

(76) Inventors: Oliver Hasselwander, Landau (DE); Klaus Kramer, Landau (DE); Kai-Uwe Baldenius, Ludwigshafen (DE); Martin Jochen Klatt, Bad Durkheim (DE); Loni Schweikert, Altrip (DE)

Correspondence Address: KEIL & WEINKAUF, 1350 CONNECTICUT AVENUE, N.W., WASHINGTON, DC, 20036, US

(21) Appl. No.: 10/363,658

(22) PCT Filed: Sep. 11, 2001
(86) PCT No.: PCT/EP01/10501

(30) Foreign Application Priority Data
Sep. 12, 2000 (DE) 100450598

(51) Int. Cl. A61K 31/385; A61K 31/202

(52) U.S. Cl. 514/440; 514/560

(57) ABSTRACT

The present invention relates to the use of lipoic acid, a physiologically acceptable derivative or salt thereof and one compound of the formula I

H₃C-(CH₂)ₙ₁-(CH═CH)ₙ₃-(CH₂)ₙ₂—COOH I

in which n1, n2 have, independently of one another, the value of an integer from 3 to 9, n3 has the value of an integer from 2 to 6, and the number of carbon atoms is 18, 20 or 22, a physiologically acceptable derivative or salt thereof, for nutritional supplementation, in functional foods and for therapeutic purposes in the treatment of diabetic disorders. Compositions having a corresponding active ingredient combination, and compositions in the form of commercial packs with corresponding combination products or single-drug products for combined use are also described.

The present invention relates to the use of lipoic acid and conjugated fatty acids for nutritional supplementation, in functional foods and for therapeutic purposes in the treatment of diabetic disorders, compositions having a corresponding combination of active ingredients, and compositions in the form of commercial packs having corresponding combination products or monoproducts for combined use.

Recent estimates are that there are about 135 million patients suffering from diabetes mellitus in the work. The number is expected to rise to about 300 million by 2025. In the USA alone, about 15.7 million people, which is 5.9% of the population, suffer from diabetes mellitus. This includes about 5.4 million with undiagnosed disease.

Diabetes mellitus, normally referred to just as diabetes, is a disorder of carbohydrate metabolism. Essentially two forms of this disease are found: type I diabetes resulting from insulin deficiency and therefore also referred to as insulin-dependent diabetes mellitus (IDDM), and type II diabetes resulting from a reduced effect of insulin, therefore also referred to as non-insulin-dependent diabetes mellitus (NIDDM). Type I usually becomes manifest in children and adolescents and requires life-long insulin administration for treatment. However, more than 90% of all the cases are type II, which usually becomes manifest in adulthood. Treatment of this type, which is essentially attributable to insulin resistance, usually takes place with special diabetic diet and antidiabetics with regular monitoring of the blood glucose level. Only in rare cases is insulin treatment of type II indicated.

The principal active ingredients employed for symptomatic treatment of hyperglycemia comprise hypoglycemic active ingredients, which include in particular the sulfonylureas, and antihyperglycemic active ingredients, which include the biguanidines, the α-glucosidase inhibitors and the insulin sensitizers, particularly the thiazolidinediones.

Sulfonylureas such as tolbutamide, chlorpropamide, glyburide and similar sulfonylureas of the first and second generations lower the plasma glucose level primarily by stimulating insulin secretion. The high risk, associated therewith, of developing a sulfonylurea-induced hypoglycemia is a problem, leading to the necessity for many, especially elderly, patients to be hospitalized.

Biguanides such as metformin reduce hepatic glucose production. However, these active ingredients are contraindicated for patients with renal or hepatic dysfunction and in cases of alcoholism. In addition, gastrointestinal side effects are common.

α-Glucosidase inhibitors such as acarbose competitively inhibit the hydrolysis of saccharides. These active ingredients also frequently cause unwanted side effects.

Thiazolidinediones such as torglitazone improve the insulin sensitivity of the skeletal muscle and reduce the hepatic glucose output. However, the problem with these active ingredients is the possible idiosyncratic hepatotoxicity. Thus, despite the wide variety of antidiabetics available, there is a need for further treatment options for diabetes mellitus.

Lipoic acid is a coenzyme in the oxidative decarboxylation of α-keto acids and is found in virtually every cell in the body. The antiinflammatory, analgesic and cytoprotective properties of lipoic acid, and its antioxidant effect, make it an interesting active ingredient for pharmacy, cosmetics, food science and adjacent areas (Biothiols in Health and Disease, edited by Packer L. and Cadenas E., Marcel Dekker Inc., New York, Basle, Hong Kong). Thus, their studies on diabetic patients in which administration of lipoic acids showed an effect have been reported. For example, Jacob et al., Arzneim.-Forsch./Drug Res. 45 (II) No. 8 (1995) 872-874 describe a distinct improvement in the glucose utilization of patients with type II diabetes after a single parenteral dose of 1,000 mg of lipoic acid. Similar results have been reported with chronic parenteral administration (Jacob et al., Exp. Clin. Endocrinol. Diabetes 104 (1996) 284-288). In a study of the treatment of diabetic neuropathy with lipoic acid (ALADIN) symptomatic complaints decreased with intravenous administration of 600 mg of lipoic acid a day for 3 weeks (Ziegler et al., Diabetologia (1995) 38: 1425-1433). However, in a recent continuation of this study on the symptomatic treatment of diabetic polyneuropathy with lipoic acid (ALADIN III), no effect on neuropathic symptoms distinguishable from placebo was found, although the 3-week intravenous and subsequent 6-months oral treatment with lipoic acid appeared to have a beneficial effect on neuropathic deficits (Zieler et al., Diabetes Care 22: 1296-1301, 1999). In the DEKAN study, no significant change in cardiovascular autonomic symptoms was observable in NIDDM patients with the 800 mg of lipoic acid each day for 4 months (Zieler et al., Diabetes Care 20 (1997) 369-373). It was found in a recent multicenter study of patients with type II diabetes that oral administration of 600 mg of lipoic acid once to 3 times a day was able to influence the insulin sensitivity (Jacob et al., Free Radical Biology & Medicine, Vol. 27, Nos. 3/4, 309-314, 1999 and BioFactors 10 (1999) 169-174).

In Germany, the lipoic acid-containing products are currently listed for the treatment of abnormal sensation associated with diabetic polyneuropathy. Formulations of solid salts of lipoic acid are proposed in U.S. Pat. No. 5,990,152. U.S. Pat. No. 5,994,393 relates to another modification of lipoic acid. Useful lipoic acid analogs are proposed in WO 99/45922. Combinations of lipoic acid and vitamins for producing pharmaceuticals are described in EP 0 572 922 A1. A combination of lipoic acid as biocompatible disulfide with an essential fatty acid, in particular gamma-linolenic acid, arachidonic acid, stearidonic acid, eicosapentaenoic acid and docosahexaenoic acid, and, where appropriate, other essential nutrients, inter alia for the treatment of diabetes and diabetic complications, is mentioned in WO 99/04782. According to EP 0 218 460, specifically diabetic neuropathies and other long-term complications of diabetic origin can be treated with gamma-linolenic acid and metabolites thereof.

Besides essentially fatty acids, however, other fatty acids which are nonessential per se may be important for the human or animal body. For example, anticarcinogenic, antiarteriosclerotic and immunostimulant activity has been ascribed to the conjugated linoleic acids discovered in the late 1980s. These comprise a group of linoleic acid isomers, e.g. with two conjugated double bonds located at positions 9 and 11 or 10 and 12. The use of conjugated linoleic acids for treating diabetes was originally proposed in WO 99/29317.

It has now been found that certain combined uses of lipoic acid with conjugated fatty acids make it possible effectively to treat diabetic disorders and thus represent an ideal nutritional supplementation, confer on foods a beneficial effect on health, and have a high therapeutic value.

The present invention therefore relates to the use of at least one lipoic acid, a physiologically acceptable derivative or salt thereof and at least one compound of the formula I

H₃C-(CH₂)ₙ₁-(CH═CH)ₙ₃-(CH₂)ₙ₂—COOH I

in which n1, n2 have, independently of one another, the value of an integer from 3 to 9, n3 has the value of an integer from 2 to 6, preferably 2 or 3, and the total number of carbon atoms is 18, 20 or 22, preferably 18, a physiologically acceptable derivative or salt thereof, for the treatment of diabetic disorders.

The novel treatment represents a combination therapy, i.e. the use of lipoic acids, physiologically acceptable derivatives or salts thereof—also referred to hereinafter for simplicity as “lipoic acid component” and the use of compounds of the formula I, physiologically acceptable derivatives or salts thereof—referred to hereinafter for simplicity as CA component (for conjugated acid component)—takes place in a situation which is, in particular, therapeutically appropriate, especially in relation to optimal efficacy. Thus, the lipoic acid component and the CA component can in principle be administered together in one formulation or separately in at least two different formulations. The latter possibility includes both administration simultaneously, i.e. taking place at essentially identical times or in direct succession, and administration with a time lag, i.e. at different times. A particular embodiment of the administration with a time lag is achieved by alternate administration of the two components, for example with an early/late daily rhythm.

The present invention thus relates both to the use of at least one lipoic acid, a physiologically acceptable derivative or salt thereof and the use of at least one compound of the formula I, a physiologically acceptable derivative or salt thereof, for the CA-assisted treatment or for the lipoic acid-assisted treatment of diabetic disorders. In this sense, the invention relates to compositions for the treatment of diabetic disorders which are based on the combination of at least one lipoic acid, a physiologically acceptable derivative or salt thereof and at least one compound of the formula I, a physiologically acceptable derivative or salt thereof, and, where appropriate, other active ingredients, where the active ingredient components, in particular the lipoic acid and CA components, can be formulated together or separately.

The term “lipoic acid” refers according to the invention to 5-(1,2-dithiolan-3-yl)valeric acid, also called thioctic acid, of the formula II

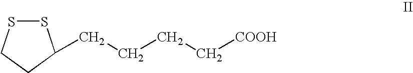

including the optical isomers covered by this formula, both as mixtures, e.g. racemates, and in pure form, e.g. R or S enantiomers. The preferred isomer is (R)-5-(1,2-dithiolan-3-yl)-valeric acid of the formula III

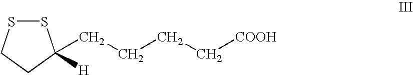

Lipoic acid mixtures with an (R) enantiomeric excess (ee) of at least 40% are preferred. The (R) enantiomeric excess is preferably at least 80%, in particular at least 98%.

Lipoic acid derivatives include, in particular, synthesis precursors and metabolites of lipoic acid, i.e. especially dihydrolipoic acid. Other metabolites which should be mentioned are lipoamide, lipoyllysine, di-6,8-bisnorlipoic acid and tetranorlipoic acid. Other suitable lipoic acid derivatives are, for example, the esters, thioesters and amides of lipoic acid with amino alcohols, amino thiols and diamines which are described in WO 99/45922 as lipoic acid analogs of the formula (I) and which are incorporated in the present application by reference. Corresponding to the statements about lipoic acid, the respective optical isomers of the derivatives also belong therewith.

The physiologically acceptable salts of lipoic acid or lipoic acid derivatives are in the present case preferably base addition salts.

The base addition salts include salts with inorganic bases, for example metal hydroxides or carbonates of alkali metals, alkaline earth metals or transition metals, or with organic bases, for example ammonia or basic amino acids such as arginine and lysine, amines, e.g. methylamine, dimethylamine, trimethylamine, triethylamine, ethylamine, diethylamine, ethylenediamine, ethanolamine, diethanolamine, 1-amino-2-propanol, 3-amino-1-propanol or hexamethylenetetramine, saturated cyclic amines with 4 to 6 ring carbon atoms, such as piperidine, piperazine, pyrrolidine and morpholine, and other organic bases, for example N-methylglucamine, creatine and tromethamine, and quaternary ammonium compounds such as tetramethylammonium and the like.

Salts with inorganic bases are preferred, e.g. Na, K, Mg, Ca, Zn, Cr and Fe salts.

The compounds of the formula I are polyunsaturated fatty acids with conjugated double bonds (conjugated fatty acids). They particularly include compounds of the formula IV

H₃C-(CH₂)ₙ₁—CH═CH—CH═CH-(CH₂)ₙ₂—COOH IV

in which n1, n2 have, independently of one another, the value of an integer from 3 to 9, and the total of (n1+n2) is 12. These have, like linoleic acid, 18 carbon atoms and 2 double bonds (C₁₈:2). In contrast to linoleic acid, the double bonds are conjugated and may also have the trans configuration besides the cis configuration present in the linoleic acid. These compounds of the formula IV, which are to be referred to as conjugated linoleic acids (CLA), therefore represent isomers of linoleic acid differing from this essential fatty acid by the position and/or cis,trans configuration. They are accordingly positional isomers and/or geometric isomers. Other useful conjugated fatty acids include, in particular, calendulic acid (8t,10t,12c-octadecatrienoic acid).

Compounds of the formula IV include, in particular, octadecadi-8,10-enoic acids (n1,n2=6), octadecadi-9,11-enoic acids (n1=5; n2=7), octadecadi-10,12-enoic acids (n1=4; n2=8) and octadecadi-11,13-enoic acids (n1=3; n2=9). Of these isomers, those preferably employed are the 9,11 and 10,12 isomers.

With regard to the geometric configuration, cis,trans and trans,cis isomers are preferred to cis,cis and trans,trans isomers, for example, 9c,11t and 9t,11c isomers to 9c,11c and 9t,11t isomers, and in a corresponding application the cis,trans and trans,cis isomers of the 8,10-, 10,12- and the 11,13-dienoic acids.

In a particular embodiment, the CA component comprises at least one compound of the formula IV which is selected from the 9c,11t and 10t,12c isomers of octadecadienoic acid.

The use of one isomer as CA component may be expedient. On the other hand, it may for practical reasons be advantageous to use two or more of the aforementioned isomers. It is moreover possible for the isomers to differ from one another in the position and/or geometric configuration of their double bonds.

The proportion of 9,11 and/or 10,12 isomers in the total weight of the CA component is advantageously at least 50% by weight, preferably at least 70% by weight and in particular at least 90% by weight. According to another advantageous aspect, the proportion of compounds of the formula IV other than the 9,11 and/or 10,12 isomers is in each case less than 5% by weight, preferably less than 2% by weight and, in particular less than 1.5% by weight. In one embodiment of the present invention, 9,11 and 10,12 isomers are used in combination. It is possible in this case also for proportions of 8,10 and/or 11,13 isomers to be present. It is preferred in this case to use relatively large amounts of 9,11 and 10,12 isomers than of 8,10 and/or 11,13 isomers. It is advantageous for the ratio of the total amounts of 9,11 and 10,12 isomers to the total amount of 8,10 and 11,13 isomers to be at least 3:2, preferably at least 10:1 and, in particular, at least 50:1. It is particularly preferred for the proportion of 8,10 and/or 11,13 isomers in each case to be less than 1% by weight based on the total weight of the CA component.

In a particular embodiment, CA combinations essentially formed by 9,11 and 10,12 isomers are used. For this purpose, the ratio of 9,11 and 10,12 isomers to the residual proportion of 8,10 and/or 11,13 isomers can be chosen to be greater than 9:1, preferably greater than 9.5:0.5 and, in particular, greater than 9.9:0.1.

Within the scope of the embodiment described above, each of the positional isomers can comprise one or more of the possible geometric isomers. It is thus possible in fact to use all 16 isomers, especially within the scope of the preferred and advantageous distributions of individual positional isomers described above. In this connection it is advantageous for the respective proportion of cis,trans and trans,cis isomers to predominate in relation to cis,cis and/or trans,trans isomers. Preferred ratios of cis,trans and trans,cis isomers to cis,cis and/or trans,trans isomers are at least 5:1, preferably at least 10:1 and in particular, at least 25:1.

The combined use of a plurality of isomers usually takes place as mixture.

Derivatives of compounds of the formula I include in particular esters which can be hydrolyzed under physiological conditions, and metabolites of these compounds, in particular derivatives, which are unsaturated 3 times, 4 times, 5 times or 6 times and are extended where appropriate by 2 or 4 carbon atoms, of compounds of the formula IV. Derivatives of this type can be obtained, for example, by the action of desaturases and/or elongases. Hydroxylated forms of compounds of the formula I are also included in the derivatives. Preferred esters are based on compounds of the formula I with alcohols which are also to be found in naturally occurring fatty acid esters, or with C₁-C₈-, preferably C₂-C₄-alkanols. Thus, ester derivatives of the compounds of the formula I which should be mentioned in particular are glycerides and phospholipids, it being possible for one or two acid residues not covered by formula I to be bound in the tri- and diglycerides and in phospholipids. The alkyl esters which should be particularly mentioned are the ethyl esters. Otherwise, the above statements concerning the isomers apply correspondingly to the derivatives.

The physiologically acceptable salts of compounds of the formula I and of derivatives thereof in the present case are preferably base addition salts, in particular the salts indicated in connection with lipoic acid.

Compounds of the formula I can be prepared in a manner known per se. Some of them are also available for purchase. In many cases they are mixtures of isomeric compounds of the formula I, whose composition may vary depending on the mode of production and the starting material and, where appropriate, measures taken to enrich and/or remove one or more isomers. Mixtures of this type may also comprise other constituents, in particular other fatty acids, especially linoleic acid, oleic acid, palmitic acid, stearic acid and other lipids or lipoids.

It is possible, for example, to prepare compounds of the formula I and, in particular, compounds of the formula IV from the unconjugated fatty acids, in particular linoleic acid or linoleic acid-containing mixtures. Chemical and enzymatic processes are known. Examples which may be mentioned are isomerization reactions of unconjugated fatty acids, in particular linoleic acid, under alkaline conditions or with enzymatic catalysis by transisomerases, for example the transisomerase from Butyrivibrio fibrisolvens. Fermentative processes, e.g. fermentation of lactobacillus, clostridium and bifidobacteria, are also suitable.

Linoleic acid or linoleic acid-containing mixtures can be obtained in a manner known per se. Some of them can likewise be obtained by purchase. Examples which may be mentioned are soybean oil, linseed oil and, especially, sunflower oil and safflower oil. Further statements on the preparation of compounds of the formula I and, in particular, compounds of the formula IV are to be found, for example, in WO 99/29317, U.S. Pat. No. 6,015,833 and U.S. Pat. No. 6,060,514 and the prior art mentioned therein.

Besides the lipoic acid and CA components, the novel treatment may include other active ingredients. These active ingredients may be, in particular, those whose effect is similar to or supplements the effect mediated by lipoic acid and CA. Thus, it may be advantageous to administer, in addition to the novel combination, antioxidants, insulin, antidiabetics, especially the hypoglycemics and antihyperglycemics described at the outset, and similar active ingredients. Vitamins, cofactors, trace elements, especially Cr and Zn, minerals, amino acids and other essential nutrients may also be expedient. For practical reasons, in many cases other fatty acids, where appropriate in the form of glycerides, will also be included. Polyunsaturated fatty acids, especially ω-3- and ω-6-PUFA, e.g. arachidonic acid and, in particular, docosahexaenoic acid and/or eicosapentaenoic acid; phospholipids, especially phosphatidylcholine, phosphatidylserine and phosphatidylethanolamine; antioxidants, especially vitamin E and C, flavonoids, tocotrienols, carnitine etc. are preferably administered together with lipoic acid and compounds of the formula I.

A particular embodiment of the present invention is based on the combination of a lipoic acid, especially R-lipoic acid, a physiologically acceptable derivative or salt thereof with 9c,11t and/or 10t,12c isomers of octadecadienoic acid, or physiologically acceptable derivatives or salts thereof.

The invention encompasses within the scope of therapeutic uses a nutritional supplementation, a dietary nutritional strategy or in the area of fortified foods (functional foods) a treatment of individuals.

In the area of nutritional supplementation, the intake ensured by the normal diet is supplemented by an active ingredient combination of the invention. In this sense, the active ingredient combination of the invention is also to be regarded as a nutrient combination. The purpose of this nutritional supplementation may be to compensate for corresponding dietary deficiencies or ensure an intake of these active ingredients which is above the amount ensured with the normal diet. Thus, the use according to the invention for nutritional supplementation also serves physiological dietary purposes, in particular the treatment of corresponding deficiency symptoms and alteration of particular states of an individual, which can be respectively compensated and brought about with a nutritional supplementary intake of the active ingredient combination of the invention. The deficiency symptoms and alterable states include the disorders which can be treated, and effects which can be achieved, according to the invention which are listed below.

The use according to the invention for therapeutic purposes relates in particular to the treatment of diabetic disorders. By these are meant disorders of carbohydrate metabolism. It is a syndrome characterized by hyperglycemia and associated with reduced insulin secretion and/or insulin effect. It includes, in particular, type I diabetes and the type II diabetes which can be treated in particular according to the invention, and other disorders of diabetic origin. The diabetic disorders which can be treated according to the invention also occasionally include disorders which may lead to diabetes without it being necessary for a hyperglycemic state to exist at the time of treatment. These include, for example, an insulin resistance and a reduced glucose tolerance. It is possible in particular to treat states of reduced glucose-stimulated insulin secretion.

Disorders of diabetic origin include in particular diseases attributable to hyperglycemic and/or hyperinsulinemic states. These are, in particular, micro- and macrovascular complications leading to diseases of nerves and blood vessels, such as neuropathies, nephropathies and retinopathies or atherosclerosis, and the sequelae attributable thereto, such as cataract, blindness, renal failure and/or amputations. The neuropathies which can be treated according to the invention include in particular polyneuropathies.

Preferred embodiments of the present invention are directed at the treatment of insulin resistance, in particular insulin resistance associated with type II diabetes.

Another preferred embodiment of the present invention is directed at the treatment of hyperglycemias, in particular insulin resistance-related hyperglycemias.

In adults, the novel treatment gains importance with increasing age. The treatment has particular advantages in the group of those over 40 years of ago and, in particular, those over 50 years of age. Obese individuals represent another group of individuals which can be treated advantageously.

Diseases to be treated according to the invention are frequently characterized by a progressive development, i.e. the states described above change over the course of time; ordinarily, the severity increases and it is possible where appropriate for states to interchange or other states to be added to previously existing states. In particular, disorders of diabetic origin increase with the duration of hyperglycemic and/or hyperinsulinemic states. Thus, preventive treatment of disorders of diabetic origin, in particular of sequelae, such as cataract, blindness, renal failure and amputation, represents a particularly valuable aspect of a novel treatment.

It is possible by the novel treatment to treat a plurality of signs, symptoms and/or dysfunctions associated with the aforementioned disorders and states. These include, for example, blurred vision, drowsiness, nausea, symptoms of coronary vessel disease, limping, gangrene, retinal detachment or hemorrhage, cataract, blindness, a nephrotic syndrome, renal failure, sensory deficits, deafness, stabbing or burning pain, abnormal sensation in the extremities, amputation, states of exhaustion, deep-seated pain, hypersensitivity.

One aspect of a treatment in the sense according to the invention relates to the treatment of acute or chronic disorders, states, signs, symptoms and/or dysfunctions, in particular of hyperglycemic and/or hyperinsulinemic states; one purpose of this treatment is to eliminate the disorders, regulate the states, or alleviate the signs, symptoms and/or dysfunctions, especially regulate and, in particular, reduce the blood glucose and/or blood insulin levels or stimulate the glucose-induced insulin secretion and/or increase the insulin-mediated glucose uptake. According to a particular aspect, it may be the purpose of the treatment to reduce the activity of the redox factor NFKB which has proinflammatory effects, and/or to activate peroxisome proliferator-activated receptors (PPAR). A further aspect relates to a preventive treatment (prophylaxis), in particular in relation to the aforementioned disorders of diabetic origin; one purpose of this treatment is to avert the occurrence of the disorders, states, signs, symptoms and/or dysfunctions, which also includes a postponement of the occurrence. The treatment may aim at being symptomatic, for example as symptom suppression. It may take place short-term, aim at being medium-term or else comprise a long-term treatment, for example as part of a maintenance therapy.

The novel use of the described active ingredients comprises a method within the scope of the treatment. This entails the individual to be treated, preferably a mammal, in particular a human, and also an agricultural or domestic animal being administered an effective amount of lipoic acid component and an effective amount of CA component, usually formulated in accordance with the practice of pharmacy, veterinary medicine or food technology. Whether such a treatment is indicated, and the form it is to take, depend on the individual case and may be subject both to specialist medical (usually objective diagnosis) and nonspecialist assessment (usually self-diagnosis) which may take account of the signs, symptoms and/or dysfunctions present, the risks of developing certain signs, symptoms and/or dysfunctions, and other factors.

The treatment usually takes place by single or multiple daily dosage, where appropriate together or alternately with other active ingredients or active ingredient-containing products, so that an individual to be treated is administered a daily dose of about 1 mg to 5 g, preferably from about 10 mg to 1 g, of lipoic acid and of about 10 μg to 10 g, preferably from about 10 mg to 5 g, of at least one compound of the formula I, on oral administration, and of about 5 mg to 1 g of lipoic acid, and about 1 mg to 1 g of at least one compound of the formula I, on parenteral administration.

The quantities and proportions of active ingredients are based on the active ingredients so that an appropriate conversion is necessary for salts and derivatives.

The invention also relates to the production of compositions for the treatment of an individual, preferably a mammal, in particular a human or agricultural or domestic animal.

The present invention therefore also relates in one aspect to compositions comprising

i) at least one lipoic acid, a physiologically acceptable derivative or salt therefore, and

ii) at least one compound of the formula I, a physiologically acceptable derivative or salt thereof, and

where appropriate at least one other active ingredient and a formulation base.

Novel compositions are therefore based on an active ingredient combination and, where appropriate, a formulation base.

The compositions include, in particular, pharmaceutical compositions, nutritional supplements and foods, in particular functional or dietetic foods. The novel foodstuffs and food supplements have, besides a predominantly nutrition-related function, additionally an active ingredient-related function which relates in particular to the active ingredient combination of the invention. They are therefore referred to as functional or dietetic foods or nutrients. Nutritional supplements serve to supplement the daily diet with the active ingredient combination of the invention, with the nutrition-related function of the nutritional supplement on its own becoming of less importance.

The active ingredient combination for the purpose of the invention comprises as active ingredient component i) at least one lipoic acid, a physiologically acceptable derivative or salt thereof. Mixtures of these forms are possible, but will be considered only in certain cases. In a particular embodiment, the active ingredient component i) consists of lipoic acid, preferably at least 90% by weight and, in particular, at least 99% by weight of the (R) enantiomer, where the percent by weight data are based on the total weight of active ingredient component i).

The active ingredient combination additionally comprises for the purpose of the invention as active ingredient component ii) at least one compound of the formula I, a physiologically acceptable derivative or salt thereof. The active ingredients mentioned above as particular compounds of the formula I are preferred, especially compounds of the formula IV. Mixtures of a plurality of compounds of the formula I are usually involved. In a particular embodiment, the active ingredient component ii) consists of 9c,11t and/or 10t,12c isomers of octadecadienoic acid.

The active ingredient combination for the purpose of the invention may additionally comprise as active ingredient component iii) other active ingredients, for example the active ingredients mentioned in this connection above.

The proportion of active ingredient combination in the formulation is larger than the proportion present where appropriate in natural sources, especially foods. In this sense, the novel compositions are enriched in relation to the active ingredient combination. The proportion of active ingredient combination of i) and ii) in the formulation is preferably greater than about 0.05% by weight, advantageously greater than about 0.1% by weight and, in particular, greater than about 0.5% by weight. In the case of a pharmaceutical composition, the proportion is usually about 1 to 60% by weight, preferably about 5 to 35% by weight and, in particular, about 10 to 30% by weight, and in the case of a of a nutritional supplement and especially in the case of foods where appropriate correspondingly lower if the formulation is given in larger amounts.

Unless otherwise indicated, data in percent by weight are based on the total weight of the formulation.

The formulation base for novel formulations comprises physiologically acceptable excipients. Physiologically acceptable excipients are those known to be usable in the sectors of pharmacy, food technology and adjacent areas, in particular the excipients listed in relevant pharmacopeias (e.g. DAB, Ph. Eur., BP, NF), and other excipients whose properties do not stand in the way of physiological use. Excipients for a purpose of the invention may also have a nutritional value and are therefore generally used as food component. They may also include nutrients, especially essential nutrients.

Suitable excipients may be: lubricants; wetting agents; emulsifying and suspending agents; preservatives; antioxidants; antiirritants; chelating agents; tablet coating aids; emulsion stabilizers; film formers; gels formers; odor-masking agents; masking flavors; resins; hydrocolloids; solvents; solubilizers; neutralizers; permeation promoters; pigments; quaternary ammonium compounds; refatting and superfatting agents; ointment, cream or oil bases; silicone derivatives; spreading aids; stabilizers; sterilants; suppository bases; tablet excipients such as binders, fillers, lubricants, disintegrants or coatings; propellants; diesiccants; opacifiers; thickeners; waxes; plasticizers; white oils. An arrangement concerning this is based on specialist knowledge as described, for example, in Fiedler, H. P., Lexikon der Hilfsstoffe für Pharmazie, Kosmetik und angrenzende Gebiete, 4th edition, Aulendorf: ECV-Editio-Cantor-Verlag, 1996.

Food components usually comprise one or more amino acids, carbohydrates or fats and are suitable for the human and/or animal diet. They comprise individual components, frequently vegetable but also animal products, especially sucrose, where appropriate in the form of syrups, fruit preparations such as fruit juices, nectar, fruit pulps, purees or dried fruit, for example apple juice, grapefruit juice, orange juice, apple purée, tomato sauce, tomato juice, tomato purée; cereals products such as wheat flour, rye flour, oat flour, corn flour, barley flour, spelt flour, corn syrup and starches from said cereals; dairy products such as milk protein, whey, yoghurt, lecithin and lactose.

Essential nutrients include, in particular, vitamins, provitamins, minerals, trace elements, amino acids and fatty acids. Essential amino acids which may be mentioned are isoleucine, leucine, lysine, methionine, phenylalanine, threonine, tryptophan and valine. They also include semiessential amino acids which must be given, for example, in periods of growth or deficiency states, such as glutamine, arginine, histidine, cysteine and tyrosine. Trace elements which may be mentioned are: essential trace elements and minerals which have been proved to be necessary for humans and deficiency of which leads to manifestation of clinical symptoms: iron, copper, zinc, chromium, selenium, calcium, magnesium, potassium, manganese, cobalt, molybdenum, iodine, silicon, fluorine. Likewise elements whose function in humans is as yet inadequately verified: tin, nickel, vanadium, arsenic, lithium, lead, boron. Fatty acids essential for humans which may be mentioned are: linoleic acid and linolenic acid, ARA (arachidonic acid) and DHA (docosahexaenoic acid) for babies and possibly EPA (eicosapentaenoic acid) and DHA also for adults. A comprehensive list of minerals are to be found in “Referenzwerte für die Nährstoffzufuhr”, 1st edition, Umschau Braus Verlag, Frankfurt am Main, 2000, edited by the Deutsche Gesellschaft für Ernährung.

The total of active ingredient component and formulation base is usually 100% by weight.

Examples of suitable formulations for nutritional supplementation are capsules, tablets, pills, powder sachets, liquid ampuls and bottles with integral droppers, as well as the drug forms mentioned below.

Examples of suitable pharmaceutical formulations are solid drop forms such as oral powders, dusting powders, granules, tablets, especially film-coated tablets, pastilles, sachets, cachets, sugar-coated tablets, capsules such as hard and soft gelatin capsules, suppositories or vaginal drug forms, semisolid drug forms such as ointments, creams, hydrogels, pastes or plasters, and liquid drug forms such as solutions, emulsions, especially oil-in-water emulsions, suspensions, for example lotions, preparations for injection and infusion, eye drops and ear drops. It is also possible to use implanted delivery devices for administering active ingredients of the invention. Liposomes or microspheres may also be used.

Foodstuff formulations usually have the normal form and are preferably marketed in the form of infant food, breakfast products, especially in the form of mueslis or bars, sports beverages, complete meals, especially in the framework of completely balanced diets, dietary products such as diet beverages, diet meals and diet bars.

The formulations are preferably administered by the oral route but they can also, especially in the drugs sector, be administered by the rectal, transdermal, subcutaneous, intravenous, intramuscular or intranasal route.

For producing the compositions, the active ingredients are usually mixed or diluted with a suitable excipient. Excipients may be solid, semisolid or liquid materials serving as vehicle, carrier or medium for the active ingredient. Admixture of other excipients takes place, if necessary, in a manner known per se. It is possible to carry out shaping steps, where appropriate in conjunction with mixing processes, e.g. a granulation, compression and the like.

The active ingredient components can, in particular, be formulated together. However, they may also be initially processed separately and subsequently combined in a compartmented, e.g. multilayer, drug form. It is possible in this way to take account of possible active ingredient incompatibilities and different active ingredient properties such as bioavailability, stability, solubility and the like.

The present invention further relates to compositions in the form of a commercial pack having at least one composition based on

i) at least one lipoic acid, a physiologically acceptable derivative or salt thereof, and/or

ii) at least one compound of the formula I, a physiologically acceptable derivative or salt thereof,

where appropriate with instructions for the therapeutic use of lipoic acid, physiologically acceptable derivatives or salts thereof in combination with compounds of the formula I, physiologically acceptable derivatives or salts thereof.

One embodiment of this aspect of the invention relates to commercial packs having at least one, in particular pharmaceutical, composition of the type described above with an active ingredient combination of the invention. This embodiment also encompasses commercial packs having a plurality of combination products in diverse dosages or formulations. Commercial packs of this embodiment accordingly comprise active ingredient components i) and ii) formulated together.

Another embodiment relates to commercial products having two or more, in particular pharmaceutical, compositions which are spatially separated from one another and of which at least two compositions comprise different active ingredients. These compositions may be, in particular, single-drug products, i.e. especially those with active ingredient component i) or ii). In these cases, the commercial pack contains instructions in the sense of the invention for the combined use of the compositions comprising i) and ii). Commercial packs of this embodiment accordingly comprise active ingredient components i) and/or ii) formulated separately, i.e. in the form of at least two spatially separate compositions.

Another embodiment relates to commercial packs having at least one, in particular pharmaceutical, composition based on

i) at least one lipoic acid, a physiologically acceptable derivative or salt thereof; or

ii) at least one compound of the formula I, a physiologically acceptable derivative or salt thereof.

These take the form of single-drug products. In these cases, the commercial pack contains instructions in the sense of the invention for the therapeutic use of the composition in combination with the other active ingredients which form the active ingredient combination of the invention but are not part of the commercial pack, in the form of at least one other composition. Commercial packs of this embodiment accordingly comprise part of the active ingredient combination of the invention. The part which is not contained is included as intended as part of the enclosed instructions.

It is self-evident that commercial packs of the invention may also comprise other products, in particular active ingredient-containing formulations, and comprehensive instructions also going beyond the aforementioned contents.

The present invention is explained in detail by means of the following examples without being restricted thereto.

### EXAMPLE 1

Pharmaceutical compositions
a) Soft gelatin capsules with lipoic acid and CLA
(lipoic acid 200 mg + CLA 500 mg)

| | |
|---|---|
| CLA | 500 mg |
| Lipoic acid | 200 mg |
| D/L-alpha-tocopherol | 60 mg |

### EXAMPLE 2

Functional foodstuff
a) Bar with lipoic acid and CLA
(CLA 1000 mg + 400 mg of lipoic acid/bar (60 g)

| | | |
|---|---|---|
| CLA | 1000 | mg |
| Lipoic acid | 400 | mg |
| D/L-alpha-tocopherol | 150 | mg |
| Syrup composed of | | |
| fructose | 4.2 | g |
| glucose | 12 | g |
| caramelized sugar | 3 | g |
| glycerol | 3 | g |
| Lecithin | 125 | mg |
| Hydrogenated vegetable oil | 1.2 | g |
| Roasted oat flakes | 17.975 | g |
| Puffed rice | 7 | g |
| Roasted and chopped almonds | 5.6 | g |
| Coconut flakes | 4 | g |

b) Muesli with lipoic acid and CLA
(CLA 500 mg + lipoic acid 250 mg/100 g of muesli)

| | | |
|---|---|---|
| Oat flakes | 40 | g |
| Wheat flakes | 27 | g |
| Raisins | 13 | g |
| Dried apple slices | 6 | g |
| Dried apricots | 3 | g |
| Wheat germ | 3 | g |
| Roasted and ground hazelnuts | 6 | g |
| Supplemented milk powder, containing | 7 | g |
| CLA | 500 | mg |
| Lipoic acid | 250 | mg |
| D/L-alpha-Tocopherol | 60 | mg |
| Lecithin | 200 | mg |

### EXAMPLE 3

Biological Effect

The obese Zucker rat (fa/fa) is an animal model of insulin resistance, glucose intolerance, hyperinsulinaemia and dyslipidaemia. This model was used to determine the effect of four different forms of diet (control, R-lipoic acid (R—LA), conjugated linoleic acid (CLA) and a combination of conjugated linoleic acid and R-lipoic acid) on insulin-induced skeletal muscle glucose transport activity and on glucose tolerance.

The test animals were maintained on regular chow and water ad libitum. At the age of 9-10 weeks, the animals were assigned to either a vehicle-treated control group or to intervention groups receiving either R-lipoic acid (10 mg/kg bodyweight), conjugated linoleic acid (c9,t11:t10,c12; 50:50; 0.3 g/kg bodyweight) or both. R-lipoic acid was administered by intraperitoneal injection; conjugated linoleic acid by gavage. The treatment period lasted for 18-20 days.

24 hours after the final treatment and having been restricted to 4 g chow during the previous 13 hours, animals were deeply anaesthetised with pentobarbital sodium (50 mg/kg ip). Right soleus muscles and both right and left epitrochlearis muscles were surgically removed and prepared for incubation in vitro. Muscles were prepared as described by Peth J A et al. (Am J Physiol Regulatory Integrative Comp Physiol 2000; 278: R453-R459). Glucose transport activity was then determined as described by Hendriksen and Halseth (J Appl Physiol 1994; 76: 1862-1867).

An oral glucose tolerance test was performed on all animals after 18 days of treatment. Rats were food restricted to 4 g of regular chow for 12 hours prior to the oral glucose tolerance test. All rats were administered 1 g/kg glucose by gavage. Blood samples were obtained from a cut at the tip of the tail at baseline, 30, 60, 90 and 120 min after the glucose load. Plasma samples were analysed spectrophotometrically for glucose and free fatty acids and by radioimmunoassay for insulin.

2-Deoxyglucose uptake in the epitrochlearis and soleus muscle are shown in table 1. Basal rates of 2-deoxyglucose uptake by the muscles were not different from that of the vehicle-treated group. Significant differences in the insulin-induced glucose transport activity were observed with respect to the stimulation of skeletal muscle glucose transport activity by insulin in vitro. 2-Deoxyglucose uptake in the epitrochlearis muscle and in the soleus muscle increased from a baseline value of 120 and 220 to 278 and 579 pmol/mg muscle/20 min following stimulation by insulin (p<0.05 vs. obese vehicle-treated) in the group receiving a combination of R-lipoic acid and conjugated linoleic acid. These effects were only observed in animals receiving the combination of R-lipoic acid and conjugated linoleic acid. Neither R-lipoic acid nor conjugated linoleic acid alone increased the skeletal muscle glucose transport activity following stimulation by insulin beyond the increase observed in the vehicle-treated control group.

Table 1
2-Deoxyglucose level in pmol/mg muscle

| | | | |
|---|---|---|---|
| | | + Insulin | |
| Treatment group | Basal | [5 mU/ml] | Increase |

Epitrochlearis

| | | | |
|---|---|---|---|
| Control | 127 +/− 3 | 220 +/− 8 | 93 +/− 8 |
| 10 mg/kg R-LA | 123 +/− 3 | 225 +/− 8 | 102 +/− 11 |
| 0.3 mg/kg CLA | 126 +/− 4 | 248 +/− 14 | 123 +/− 13 |
| 10 mg/kg R-LA + 0.3 mg/kg | 120 +/− 7 | 278 +/− 12 | 158 +/− 6 |
| CLA | | | |

Soleus

| | | | |
|---|---|---|---|
| Control | 229 +/− 5 | 469 +/− 13 | 240 +/− 11 |
| 10 mg/kg R-LA | 224 +/− 7 | 481 +/− 19 | 258 +/− 16 |
| 0.3 mg/kg CLA | 231 +/− 6 | 492 +/− 9 | 261 +/− 13 |
| 10 mg/kg R-LA + 0.3 mg/kg | 220 +/− 9 | 579 +/− 40 | 359 +/− 35 |
| CLA | | | |

We claim:

1. The use of at least one lipoic acid, a physiologically acceptable derivative or salt thereof and at least one compound of the formula I
H₃C-(CH₂)ₙ₁-(CH═CH)ₙ₃-(CH₂)ₙ₂—COOH I
in which n1, n2 have, independently of one another, the value of an integer from 3 to 9, n3 has the value of an integer from 2 to 6, and the number of carbon atoms is 18, 20 or 22, a physiologically acceptable derivative or salt thereof, for the preparation of an agent for the treatment of diabetic disorders.

2. The use as claimed in claim 1, wherein the compound of formula I is a compound of the formula IV
H₃C-(CH₂)ₙ₁—CH═CH—CH═CH-(CH₂)ₙ₂—COOH IV
in which n1, n2 have, independently of one another, the value of an integer from 3 to 9, and the total of (n1+n2) is 12.

3. The use as claimed in claim 1 or 2 of at least one compound of the formula I or IV in which n1 is 3, 4, 5 and n2 is 7, 8, 9.

4. The use as claimed in claim 2 or 3, where the compound of the formula IV is selected from 9c,11t-octadecadienoic acid and 10t,12c-octadecadienoic acid.

5. The use as claimed in any of the preceding claims for the treatment of type II diabetes.

6. The use as claimed in any of the preceding claims for the treatment of insulin resistance.

7. The use as claimed in any of the preceding claims for the treatment of hyperglycemia.

8. The use as claimed in any of the preceding claims for the treatment of microvascular and macrovascular disorders of diabetic origin.

9. The use as claimed in claim 9 for the treatment of diabetic neuropathy, diabetic nephropathy, diabetic retinopathy, atherosclerosis.

10. The use as claimed in any of the preceding claims for the preventive treatment of cataract, blindness, renal failure and amputations of diabetic origin.

11. A composition comprising
i) at least one lipoic acid, a physiologically acceptable derivative or salt thereof, and
ii) at least one compound of the formula I
H₃C-(CH₂)ₙ₁-(CH═CH)ₙ₃-(CH₂)ₙ₂—COOH I
in which n1, n2 have, independently of one another, the value of an integer from 3 to 9, n3 has the value of an integer from 2 to 6, and the number of carbon atoms is 18, 20 or 22, a physiologically acceptable derivative or salt thereof, and
where appropriate at least one active ingredient and a formulation base.

12. A composition as claimed in claim 11, wherein the active ingredient content is greater than 0.05% by weight.

13. Commercial pack having at least one composition based on
i) at least one lipoic acid, a physiologically acceptable derivative or salt thereof, and
ii) at least one compound of the formula I
H₃C-(CH₂)ₙ₁-(CH═CH)ₙ₃-(CH₂)ₙ₂—COOH I
in which n1, n2 have, independently of one another, the value of an integer from 3 to 9, n3 has the value of an integer from 2 to 6, and the number of carbon atoms is 18, 20 or 22, a physiologically acceptable derivative or salt thereof, and
where appropriate with instructions for the therapeutic use of lipoic acids, physiologically acceptable derivatives or salts thereof in combination with compounds of the formula I, physiologically acceptable derivatives or salts thereof.
